(19) United States
(12) Patent Application Publication
(10) Pub. No.: US 2011/0009796 A1
(43) Pub. Date: Jan. 13, 2011

(54) METHOD AND APPARATUS FOR INCREASING CONTAMINANT CLEARANCE RATES DURING EXTRACORPOREAL FLUID TREATMENT

(75) Inventors: Richard H. Tullis, Encinitas, CA (US); Harold H. Handley, Encinitas, CA (US); Paul Duffin, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: AETHLON MEDICAL, INC., San Diego, CA (US)

(21) Appl. No.: 12/810,295

(22) PCT Filed: Dec. 19, 2008
(86) PCT No.: PCT/US08/87836

§ 371 Date: Sep. 7, 2010

Related U.S. Application Data
(60) Provisional application No. 61/016,922, filed on Dec. 27, 2007.

(51) Int. Cl. A61M 1/34; B01D 63/02; B01D 15/00; B01D 61/00

(52) U.S. Cl. 604/5.02; 210/258; 210/638; 210/256

(57) ABSTRACT

An extracorporeal fluid treatment apparatus includes a separator comprising a cartridge surrounding a porous separation membrane. The membrane separates a main flow path of a cellular component of the blood from a plasma flow path. An affinity medium is disposed within the plasma flow path to bind contaminants such as viral pathogens or toxins contained within the plasma. A pump pumps the plasma through the affinity medium at an assisted flow rate preferably between 10% and 40% of the whole blood flow rate. The assisted flow rate is selected to reduce a T90% of the apparatus by at least 50% as compared to a T90% of the apparatus without the plasma pump. A method of treating blood containing contaminants includes supplying infected blood to a separator and pumping the plasma component through an affinity medium at an assisted flow rate to increase contaminant clearance.

### BACKGROUND OF THE INVENTION

1. Field of the Invention

This application relates to extracorporeal fluid treatment devices and methods. More particularly, this application relates to devices and methods for increasing contaminant clearance rates during treatment.

2. Description of the Related Art

Extracorporeal treatments provide a therapeutic modality which can be used to remove contaminants from the blood or other bodily fluids. For example, extracorporeal perfusion of plasma over protein A, plasmapheresis and lymphapheresis have all been used as immunomodulatory treatments for HIV infection, and the thrombocytopenia resulting from it (Kiprov et al. Curr Stud Hematol Blood Transfus 57: 184-197, 1990; Mittelman et al. Semin Hematol 26(2 Suppl 1): 15-18, 1989; Snyder et al. Semin Hematol 26(2 Suppl 1): 31-41, 1989; Snyder et al. Aids 5(10): 1257-1260, 1991).

Immunosorptive techniques have been proposed for the treatment of viral infections. In 1980, Terman et al. described a plasmapheresis apparatus for the extracorporeal treatment of disease including a device having an immunoadsorbent fixed on a large surface area spiral membrane to remove disease agents (U.S. Pat. No. 4,215,688). The device envisioned no method for directly treating blood and required the presence of an immunologically reactive toxic agent. U.S. Pat. No. 6,528,057 describes the removal of virus and viral nucleic acids using antibodies and antisense DNA. Plasmapheresis methods using lectins to remove viral particles have also been described (U.S. Pat. No. 7,226,429). Other plasmapheresis techniques have been described that employ antibodies to remove biological pathogens (U.S. Pat. No. 4,787,974) or chelating agents to remove heavy metals (U.S. Pat. No. 6,071,412).

There is an ongoing need for novel therapeutic approaches for the removal of contaminants such as viral particles from bodily fluids.

### SUMMARY OF THE INVENTION

One embodiment of the invention is an extracorporeal blood treatment apparatus comprising a separator comprising a cartridge surrounding at least one hollow fiber membrane, the hollow fiber membrane having a lumen, the cartridge and the hollow fiber membrane defining an extralumenal space there between, the separator having an inlet port and an outlet port in fluid communication with the lumen, and at least one plasma port in fluid communication with the extralumenal space, where the separator is configured to allow a plasma component of blood passed through the lumen to pass through the hollow fiber membrane and into the extralumenal space while preventing a cellular portion of blood passed through the lumen to pass through the hollow fiber membrane and into the extralumenal space; an affinity medium disposed external to the hollow fiber membrane, the affinity medium being configured to bind at least one selected contaminant; and a plasma pump in fluid communication with the plasma port and configured to pump the plasma component at an assisted flow rate, the assisted flow rate being selected to increase a clearance rate of the apparatus by at least two times over a clearance rate of the apparatus without the plasma pump.

In various embodiments of the extracorporeal blood treatment apparatus, the following features are present, alone or in any combination: the assisted flow rate is between 10% and 40% of a blood flow rate into the inlet port; the affinity medium comprises lectin molecules; the lectin molecules are to be selected to bind to high mannose glycoproteins; the lectin molecules are immobilized within the extralumenal space; the lectin molecules are disposed in an affinity filter in fluid communication with the plasma port and plasma pump.

Another embodiment of the invention is an extracorporeal blood treatment apparatus comprising a separator comprising a cartridge surrounding a porous separation membrane, the separation membrane configured to allow passage of a plasma component and prevent passage of a cellular component of blood passed through the separator, the separation membrane separating a main flow path of the apparatus from a plasma flow path of the apparatus; an affinity medium disposed within the plasma flow path and configured to bind at least one selected contaminant; and a plasma pump disposed along the plasma flow path and configured to pump the plasma component at an assisted flow rate, the assisted flow rate being selected to reduce a T_{90%} of the apparatus by at least 50% as compared to a T_{90%} of the apparatus without the plasma pump.

In various embodiments of the extracorporeal blood treatment apparatus, the following features are present, alone or in any combination: the assisted flow rate is between 10% and 40% of a blood flow rate into the separator; the assisted flow rate is approximately 25% of a blood flow rate into the separator; the affinity medium is disposed within the cartridge; the affinity medium is disposed in the plasma flow path external to the cartridge; the affinity medium comprises lectins selected to bind to high mannose glycoproteins.

Another embodiment of the invention is an extracorporeal blood treatment apparatus comprising means for separating whole blood into a cellular component and a plasma component; means for removing a selected viral pathogen from the plasma component; and means for pumping the plasma component through the removing means at an assisted flow rate, the assisted flow rate being between 10% and 40% of a fluid flow rate of whole blood flowing into the apparatus, the assisted flow rate being selected to increase a pathogen clearance rate of the apparatus by at least two times as compared to an apparatus having no such pumping means, and wherein said assisted flow rate results in hemolysis that is clinically acceptable.

In various embodiments of the extracorporeal blood treatment apparatus, the following features are present, alone or in any combination: the removing means is disposed within the separating means; the removing means is disposed external to the separating means, in fluid communication with the pumping means in a plasma flow path; the removing means comprises lectins configured to bind to high mannose glycoproteins; the assisted flow rate is approximately 25% of the fluid flow rate of whole blood flowing into the apparatus; the pumping means is configured to pump the plasma component out of the separating means in a direction generally normal to a main flow path through the separator.

Another embodiment of the invention is a method for extracorporeally treating whole blood containing a viral pathogen, the method comprising supplying whole blood contaminated with a viral pathogen to a separator at a whole blood flow rate so as to separate the whole blood into a cellular component and a plasma component, the separator comprising a hollow fiber membrane; pumping the separated plasma component through an affinity medium at an assisted plasma flow rate, the assisted flow rate being between 10% and 40% of the whole blood flow rate; and combining the plasma component with the cellular component downstream of the affinity medium.

In various embodiments of the method for extracorporeally treating whole blood, the following features are present, alone or in any combination: the plasma component is pumped away from the separator in a direction generally normal to a main flow path through the separator; the viral pathogen has a viral replication rate of over 10¹¹ viral copies per day and the assisted flow rate is selected to provide a T_{90%} of not more than 1 hour.

Another embodiment of the invention is, in a method of reducing viral particles and lectin binding fragments thereof in the blood of an individual infected with a virus, where the method comprises the steps of obtaining blood from the individual, passing the blood through a porous hollow fiber membrane, wherein lectin molecules are immobilized within a porous exterior portion of the membrane, and wherein the lectin molecules bind to high mannose glycoproteins, collecting pass-through blood, and reinfusing the pass-through blood into the individual, the improvement comprising separating the blood into a plasma component traveling in a plasma flow path and a cellular component traveling in a main flow path, the hollow fiber membrane separating the main flow path from the plasma flow path; providing a plasma pump along the plasma flow path; and pumping the plasma component at an assisted flow rate selected to reduce a T_{90%} of the method by at least 50% as compared to a T_{90%} of the method without the plasma pump.

In various embodiments of the method, the following features are present, alone or in any combination: the assisted flow rate is between 10% and 40% of a blood flow rate into the porous hollow fiber membrane; the virus has a viral replication rate of at least 10⁶, 10⁷, 10⁸, 10⁹, 10¹⁰, or 10¹¹ viral copies per day and the assisted flow rate is selected to provide a T_{90%} of not more than 1 hour.

### BRIEF DESCRIPTION OF THE DRAWINGS

FIG. 1 is a schematic illustration of a longitudinal cross section of an affinity cartridge.

FIG. 2 is a schematic illustration of a horizontal cross section at plane 2 in FIG. 1.

FIG. 3 is an illustration of a channel from FIG. 2.

FIG. 4 is a schematic illustration of a conventional blood treatment system using the affinity cartridge of FIG. 1.

FIG. 5 is a schematic illustration of a blood treatment apparatus according to an embodiment.

FIG. 6 is a schematic illustration of a blood treatment apparatus according to an alternative embodiment.

FIG. 7 is a graphical representation comparing the clearance rates of 100 nm mannan-coated beads in various unassisted and assisted flow configurations, shown on a linear plot.

FIG. 8 is a graphical representation comparing the clearance rates of 100 nm mannan-coated beads in various unassisted and assisted flow configurations, shown on a logarithmic plot.

FIG. 9 is a graphical representation of the rate of hemolysis of recirculating human blood.

FIG. 10 is a graphical representation comparing the hemolysis rates of various unassisted and assisted flow configurations.

FIG. 11 is a graphical representation comparing the hemolysis rates of an external affinity cartridge configuration at varying assisted flow rates.

### DETAILED DESCRIPTION OF THE PREFERRED EMBODIMENTS

The present invention relates to improved devices and methods for removing substances from infected or contaminated bodily fluids, preferably in an extracorporeal setting. The features, aspects and advantages of the present invention will now be described with reference to the drawings of various embodiments, which are intended to be within the scope of the invention herein disclosed. These and other embodiments will become readily apparent to those skilled in the art from the following detailed description of the embodiments having reference to the attached figures, the invention not being limited to any particular embodiment(s) disclosed. All references mentioned herein are hereby incorporated by reference in their entireties.

As mentioned in the Background section, plasmapheresis techniques have been described which use lectins to remove virus and toxic viral proteins from contaminated blood. Such techniques are described in U.S. Pat. No. 7,226,429.

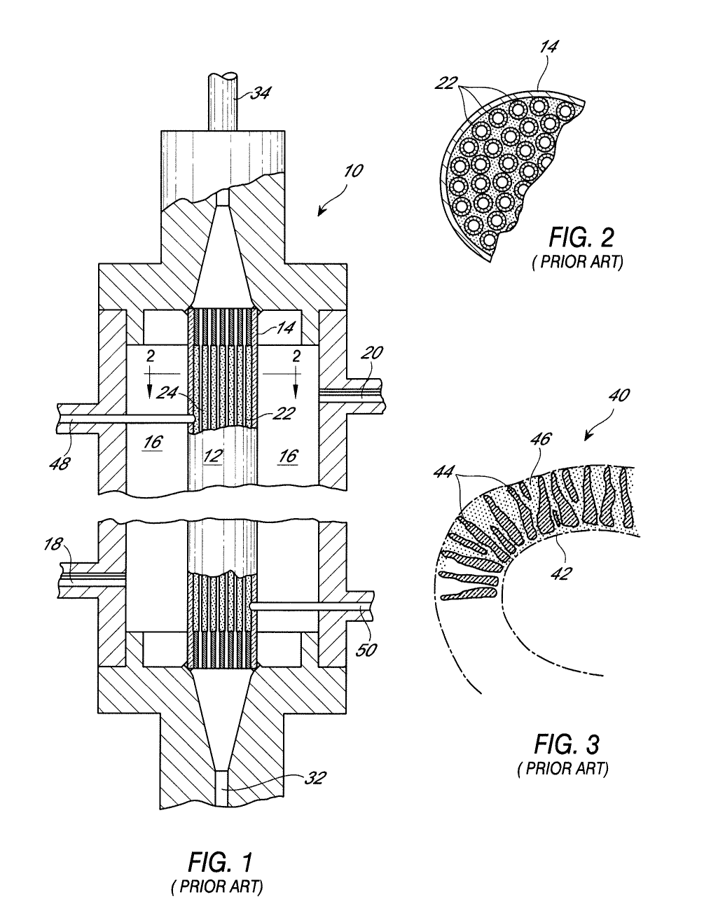

A diagram of one example of a conventional lectin-based plasmapheresis device is shown in FIG. 1. The device comprises a cartridge 10 comprising a blood-processing chamber 12 formed of interior glass or plastic wall 14. Around chamber 12 is an optional exterior chamber 16. A temperature controlling fluid can be circulated into chamber 16 through port 18 and out of port 20. The device includes an inlet port 32 for the blood and an outlet port 34 for the effluent. The device also provides one or more ports 48 and 50, for accessing the extrachannel or extralumenal space in the cartridge. The device is otherwise sealed, to prevent loss of normal plasma proteins. FIG. 2 is a schematic illustration of a horizontal cross section at plane 2 in FIG. 1. As shown in FIGS. 1 and 2, chamber 12 contains a plurality of membranes 22. FIG. 3 is a cross sectional representation of a channel 22 and shows the anisotropic nature of the membrane. As shown in FIG. 3, a hollow fiber membrane structure 40 is preferably composed of a single polymeric material which is formed into a tubular section comprising a relatively tight plasmapheresis membrane 42 and relatively porous exterior portion 44 in which lectins can be immobilized. A solution containing the lectins is loaded on to the device through port 48. The lectins are allowed to immobilize to the exterior 44 of the membrane. Unbound lectins can be collected from port 50 by washing with saline or other solutions. Alternatively, the lectins can be bound to a substrate which is loaded into the extrachannel or extralumenal space, either as a dry substance (e.g. sand), or in solution or slurry.

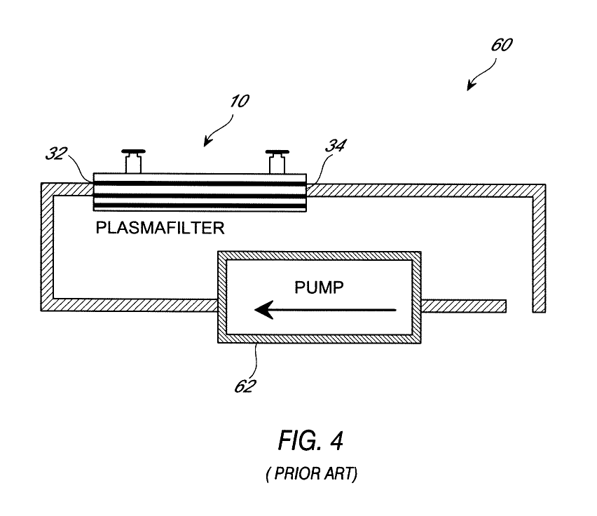

With reference to FIGS. 1-4, a conventional system 60 is illustrated which utilizes the above-described plasmapheresis device 10. Whole blood is withdrawn from a subject or other source using a pump 62, and pumped into the inlet port 32 of the device 10. As blood flows through the device 10, plasma filters through the membrane 42 and into the exterior portion 44 by convective flow, also known as Starling flow. High pressure at the proximal inlet port 32 of the device 10 forces plasma through pores in the membrane 42, allowing the plasma to contact the lectins 46 in the exterior portion 44. Blood cells and platelets are too large to pass through the pores in the membrane 42, and remain in the lumen of the hollow fibers. At the distal outlet port 34 of the cartridge, reduced luminal pressure allows the treated plasma to return to the lumen and thus to the blood as it exits the device 10. In some embodiments, the main blood flow pump 62 is downstream of the device 10.

In systems such as these, the cartridge is sealed, and relies on convective flow, or Starling flow, to drive plasma into contact with the affinity-binding agent. Unfortunately, the magnitude of Starling flow across a membrane can be relatively low for high viscosity fluids like plasma, as compared to the total fluid flow into the device. Direct measurements indicate that plasma flow rate is less than 10%, and often less than 8%, of the total blood flow rate. During use, blood elements can accumulate near and possibly clot or clog the pores, further reducing the plasma flow rate and, as a result, reducing the clearance rate.

Accordingly, embodiments of the present invention advantageously utilize a pump to increase the plasma flow rate, relative to the whole blood flow rate, in order to improve plasma contact with the affinity-binding agent. The pump assists plasma flow through a separation membrane and/or through an affinity material. In some embodiments, the affinity binding material is disposed proximate to the separation membrane, within a single separation cartridge. In other embodiments, the affinity binding material is disposed external to the separation cartridge. These and other embodiments advantageously provide contaminant clearance rates that are preferably at least two times faster than those of conventional systems, without effecting a significant change in hemolysis rates. Thus, embodiments can be used to effectively reduce viral load in patients infected with rapidly replicating viruses, such as HCV or Dengue hemorrhagic fever virus. Embodiments can also be used to provide a more rapid and efficient clearance of slower-replicating viruses such as HIV.

The term “contaminant” as used herein includes but is not limited to biological pathogens, such as viral particles and fragments thereof, exosomes, as well as toxins, chemicals, heavy metals, drugs and chemotherapeutic agents. “Contaminant” encompasses any undesirable substance which may be found in a bodily fluid.

The terms “affinity-binding material,” “affinity-binding medium,” “affinity-binding agent,” and “contaminant-binding substrate” as used herein refer to any mechanism by which a targeted contaminant may be trapped or bound and thereby removed from a fluid. “Affinity-binding material” “affinity-binding medium,” “affinity-binding agent,” and “contaminant-binding substrate” include, for example, activated charcoal, antibodies, and lectins, as well as materials in which or on which such substances may be disposed. Some examples of lectins include, without limitation, Galanthus nivalis agglutinin (GNA), Narcissus pseudonarcissus agglutinin (NPA), cyanovirin (CVN), Conconavalin A, Griffithsin and mixtures thereof.

The term “viral load” as used herein refers to the amount of viral particles or toxic fragments thereof in a biological fluid, such as blood, plasma, or bronchial or lung lavage. “Viral load” encompasses all viral particles, infectious, replicative and non-infective, and fragments thereof. Therefore, viral load represents the total number of viral particles and/or fragments thereof circulating in the biological fluid. Viral load can therefore be a measure of any of a variety of indicators of the presence of a virus, such as viral copy number per unit of blood or plasma, or units of viral proteins or fragments thereof per unit of blood or plasma.

The term “high mannose glycoprotein” as used herein for the purpose of the specification and claims refers to glycoproteins having mannose-mannose linkages in the form of α−1−>3 or α−1−>6 mannose-mannose linkages.

The terms “total fluid flow rate,” “whole blood flow rate,” and “blood flow rate” as used herein refer to the volumetric flow rate of fluid flowing into the main flow path of the device prior to any subsequent separation or treatment. The term “main flow path” refers to the flow path through the device on the same side of the membrane as the inlet.

The terms “assisted flow rate,” “secondary flow rate,” and “plasma flow rate” as used herein refer to the volumetric flow rate of the fluid passing through the membrane and flowing in a secondary flow path. The terms “secondary flow path” and “plasma flow path” refer to the flow path through the device on the opposite side of the membrane as the inlet.

The term “exposed,” as used herein in the context of a fluid being “exposed” to any type of contaminant-binding substrate, refers to any contaminated fluid or portion of a fluid contacting a contaminant-binding substrate. For example, exposure of plasma to the contaminant-binding substrate, as used herein, refers to the total amount of time the plasma is exposed to the contaminant-binding substrate and not the amount of time blood and/or plasma is processed through the device. In some embodiments, the fluid is exposed to the contaminant-binding substrate for a specific amount of time.

The time of exposure is a function of the plasma flow rate and the capacity of the contaminant-binding substrate. For example, if the whole blood flow rate of a device is 40 ml/min and the plasma assist pump is set to operate at 25% of the blood flow rate, the plasma flow rate (i.e., the assisted flow rate) is 10 ml/min. If the capacity of the contaminant-binding substrate is 10 ml, then running unprocessed blood at 40 ml/min (that is, running plasma at 10 ml/min) for 30 minutes would process 1200 ml of blood, exposing 300 ml of plasma to the contaminant-binding substrate, each ml exposed for 1 minute. If, instead of continuously processing blood, a blood pool volume of 120 ml were recirculated through the same device for 30 minutes, then 30 ml of plasma would be exposed to the contaminant-binding substrate, each ml exposed for 10 minutes. In some embodiments, the time the plasma is exposed to a contaminant-binding substrate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 minutes. In other embodiments, the time the plasma is exposed to a contaminant-binding substrate is a range defined by any two times recited above.

In a preferred embodiment, the blood flow rate into the device is about 20 ml/min to about 500 ml/min. In another preferred embodiment, the blood flow rate into the device is about 250 ml/min to about 400 ml/min. In some embodiments, the blood flow rate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml/min., or a range defined by any two of these values. In some embodiments, the plasma flow rate is, is about, is less than, is less than about, is more than, is more than about, 10%, 12%, 14%, 16%, 18%, 20%, 22%, 24%, 26%, 28%, 30%, 32%, 34%, 36%, 38%, 40%, 45%, 50%, 55%, 60%, 65%, or 70% of the blood flow rate, or a range defined by any two of these values. In some embodiments, the capacity of the device is 40 ml. Also contemplated are devices where the capacity is about, is less than, is less than about, is more than, is more than about, 3000, 2000, 1500, 1000, 750, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml, or a range defined by any two of these values.

The term “clearance rate,” as used herein, refers to the amount of time required to clear, or remove, a specified amount of contaminant from a volume of blood. For example, a system capable of reducing a viral load of 10×10⁹ copies by half (that is, to 5×10⁹ copies) in 1 hour has a clearance rate of 5×10⁹ copies/hour (or 50% per hour), and a T_{1/2} or T_{50%} value of 1 hour. A system capable of reducing a viral load of 10×10⁹ copies by 90% (that is, to 1×10⁹ copies) in 1 hour has a clearance rate of 9×10⁹ copies/hour (or 90% per hour), and a T_{90%} value of 1 hour.

In some embodiments, the viral clearance rate is, is about, is less than, is less than about, is more than, is more than about, 1×10⁴ copies/hour, 5×10⁴ copies/hour, 1×10⁵ copies/hour, 5×10⁵ copies/hour, 1×10⁶ copies/hour, 5×10⁶ copies/hour, 1×10⁷ copies/hour, 5×10⁷ copies/hour, 1×10⁸ copies/hour, 5×10⁸ copies/hour, 1×10⁹ copies/hour, 5×10⁹ copies/hour, 1×10¹⁰ copies/hour, 5×10¹⁰ copies/hour, 1×10¹¹ copies/hour, 5×10¹¹ copies/hour, 1×10¹² copies/hour, or 5×10¹² copies/hour, or a range defined by any two of these values. In some embodiments, the viral clearance rate is, is about, is less than, is less than about, is more than, is more than about, 0.1% per hour, 0.25% per hour, 0.5% per hour, 1% per hour, 2.5% per hour, 5% per hour, 10% per hour, 15% per hour, 20% per hour, 25% per hour, 30% per hour, 40% per hour, 50% per hour, 60% per hour, 70% per hour, 80% per hour, or 90% per hour, or a range defined by any of these two values. In some embodiments, continuous clearance is performed with slower clearance rates (for example, 5% per hour or less), for up to 24 hours per day over one, two, three or more days or weeks. In some embodiments, T_{1/2} or T_{50%} is, is about, is less than, is less than about, is more than, is more than about, 15, 30, or 45 minutes, or 1, 2, 3, 4, 5, 6, 7, 8, 9, 10, 11, 12, 13, 14, 15, 16, 17, 18, 19, 20, 21, 22, 23, or 24 hours, or a range defined by any two of these values. In some embodiments, T_{90%} is, is about, is less than, is less than about, is more than, is more than about, 1, 2, 3, 4, 5, 6, 7, 8, 9, 10, 11, 12, 13, 14, 15, 16, 17, or 18 hours, or a range defined by any two of these values.

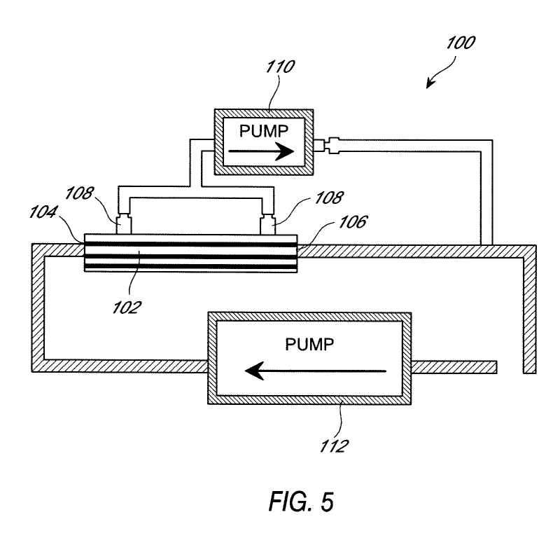

With reference to FIG. 5, an embodiment of an extracorporeal blood treatment apparatus 100 is described. The apparatus 100 includes a plasma separator 102 having an inlet port 104, an outlet port 106, a main flow pump 112, and one, two or more plasma ports 108 in fluid communication with a plasma pump 110. The plasma separator 102 preferably comprises a separation membrane surrounded by a cartridge. The separation membrane has pores sized to allow passage of the plasma component of the blood across the membrane, while preventing passage of all of, nearly or substantially all of, a majority of, or a portion of, the cellular component of the blood, including blood cells and platelets. The separation membrane thus functions to separate a main flow path, running from the inlet port 104 on one side of the membrane to the outlet port 106 of the plasma separator 102, from a plasma flow path, beginning on the other side of the membrane and running through the plasma port(s) 108 to the plasma pump 110. To allow contaminants to pass across the separation membrane along with the plasma, the pores are preferably between about 100 nm and 200 nm in diameter, or other appropriate sizes, including those described elsewhere in the specification. The inlet port 104 and the outlet port 106 are in fluid communication with the main flow path, and the plasma ports 108 are in fluid communication with the plasma flow path. To withdraw fluid in an evenly distributed flow from the extralumenal space of the cartridge and prevent accumulation and clogging of the plasma ports with matrix/substrate material, the plasma ports 108 are preferably provided with wicks configured to draw fluid from inside the separator cartridge through the plasma ports 108. The main flow pump 112 is preferably located upstream of the separator 112, but can be located downstream of the separator.

In a preferred embodiment, the separation membrane comprises one or more hollow fiber membranes. In embodiments comprising hollow fiber membranes, the inlet port 104 and the outlet port 106 are in fluid communication with the lumens of the hollow fiber membranes, which define a portion of the main flow path through the apparatus. The plasma ports 108 are in fluid communication with the extralumenal space surrounding the hollow fibers within the separator cartridge. Thus, the hollow fiber membranes separate the main flow path from the start of the plasma flow path of the apparatus 100. The hollow fiber membranes preferably have a 0.3 mm inside diameter and 0.5 mm outside diameter. In some embodiments, the outside or inside diameter is 0.025 mm to 1 mm, more preferably 0.1 to 0.5 mm, or even more preferably 0.2 to 0.3 mm. In some embodiments, the cartridge 102 includes lectins or other affinity binding materials immobilized in the extralumenal space, as described above in connection with FIGS. 1-3.

With continued reference to FIG. 5, the plasma pump 110 can comprise, for example, a negative pressure pump configured to assist the flow of plasma crossing the separation membrane and traveling through the extralumenal space containing the pathogen binding lectins, thereby increasing contact between the plasma and the lectins and increasing the clearance rate of the apparatus. As used herein, “in fluid communication” with a pump signifies that the pump is located along or within the fluid path, and includes configurations where no components of the pump contacts the fluid, such as a peristaltic pump. A pump disposed along or within a fluid path may or may not be in actual contact with the fluid moving along or through the path. Two plasma ports 108 are placed at either end of the plasma separator 102, one near the inlet port 104 and one near the outlet port 106, in order to provide more uniform flow through the extralumenal space. Of course, embodiments can include one, two or more plasma ports, depending on the particular application. As illustrated in the figure, the plasma ports 108 and the plasma pump 110 are configured to guide the plasma component through the extralumenal space in a direction generally perpendicular to the direction of the main flow path. Beyond the plasma pump 110, the plasma flow path ultimately reconnects with the main flow path to mix the treated plasma component with the cellular component for return to the patient.

Contaminant clearance rates in systems such as these are a function of the plasma flow rate through the binding material, the binding rate of the material, and the residence time in the binding material. For example, if the binding rate of a given material is relatively slow, then flow rates should be set accordingly so that the contaminant residence times are sufficient to allow for effective clearance. Increasing flow rates in such a situation will not effect an increase in the clearance rate, and may even result in dislodging bound toxins due to shear stresses. Thus, for a given contaminant and a given binding agent, an ideal range of plasma flow rates can be determined which optimizes the contaminant clearance rates. Thus, in some embodiments, the pump is configured to provide a plasma flow rate between 10% and 40% of the main fluid flow rate flowing into the apparatus 100 at inlet port 102. Preferably, the pump is configured to provide a plasma flow rate of approximately 25% of the fluid flow rate flowing into the apparatus 100. The plasma pump flow rate is preferably selected to increase the contaminant clearance rate by more than two times over that of a system relying on Starling flow alone, i.e., where the plasma flow is unassisted by a pump.

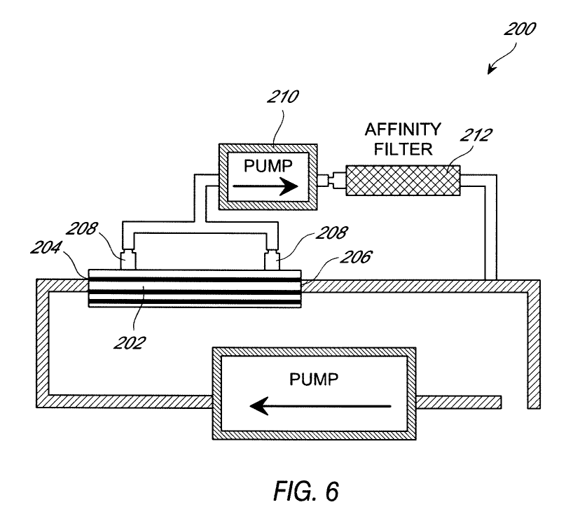

With reference to FIG. 6, an extracorporeal blood treatment apparatus 200 according to an embodiment is described. The apparatus 200 includes a plasma separator 202 having an inlet port 204, an outlet port 206, and one or more plasma ports 208 in fluid communication with a plasma pump 210. Two plasma ports 208 are placed at either end of the plasma separator 202, one near the inlet port 204 and one near the outlet port 206, as described above in connection with FIG. 5. The apparatus 200 further includes an affinity filter 212 disposed external to the plasma separator 202. The affinity filer 212 is preferably located downstream of the plasma pump 210, but can be located upstream of the pump 210.

The plasma separator 202 preferably comprises a separation membrane surrounded by a cartridge. The separation membrane has pores sized to allow passage of the plasma component of the blood across the membrane, while preventing passage of the cellular component of the blood, including blood cells and platelets. In a preferred embodiment, the separation membrane comprises one or more hollow fiber membranes as described above in connection with FIG. 5. The separation membrane functions to separate a main, e.g. blood, flow path, running from the inlet port 204 on one side of the membrane to the outlet port 206 of the plasma separator 202, from a plasma flow path, beginning on the other side of the membrane and running through the plasma port(s) 208 to the plasma pump 210. To allow contaminants to pass across the separation membrane along with the plasma, the pores can be between about 100 nm and 200 nm in diameter. In some embodiments, the pore size is between 150 and 600 nm. Additionally, in some embodiments, the pores can be about, less than, less than about, more than, or more than about, 300 nm, 400 nm, 500 nm, 600 nm, 700 nm, or a range defined by any two of the aforementioned values. Preferably, the pores are of sufficient size to allow for maximization of plasma separation from platelets at the highest flow rate possible. The inlet port 204 and the outlet port 206 are in fluid communication with the main flow path, and the plasma ports 208 are in fluid communication with the plasma flow path. To withdraw fluid in an evenly distributed flow from the extralumenal space of the cartridge, the plasma ports 208 are preferably provided with wicks configured to draw fluid from inside the separator cartridge through the plasma ports 208.

The affinity filter 212 includes an affinity binding material configured to selectively bind and remove contaminants from plasma passing through the filter 212. In a preferred embodiment, the affinity filter includes immobilized lectins configured to bind glycosylated viral particles. The plasma pump 210 is configured to assist the flow of plasma traveling across the separation membrane and through the plasma ports 208 toward the affinity filter 212, thereby increasing contact with between the plasma and the affinity binding material. As illustrated in the figure, the plasma ports 208 and the plasma pump 10 are preferably disposed so as to draw the plasma component across the separation membrane in a direction generally perpendicular to the direction of the main flow path. Beyond the affinity filter 212, the plasma flow path preferably reconnects with the main flow path to mix the treated plasma component with the cellular component, in order to be returned to the patient.

Methods for treating the blood of individuals infected with contaminants are also described. Whole blood can be withdrawn from an infected individual and supplied to a separator means configured to separate the whole blood into a cellular component and a plasma component. The separator means preferably comprises a hollow fiber membrane contained within a cartridge; however, embodiments can also include other types of separator means known in the art, such as a centrifuge, for example. The plasma component is passed through a contaminant affinity medium, such as, for example, a lectin-containing affinity matrix, which is disposed within the separator cartridge or in an external affinity cartridge. The flow rate of the plasma component through the separator, and through the affinity-binding medium, is preferably augmented by a plasma pump disposed external to the separator. The plasma is pumped at an assisted flow rate between 5% and 70%, preferably between 10% and 40%, of the whole blood flow rate. The assisted flow rate is selected to provide a contaminant clearance rate effective to reduce viral load in the infected blood. For example, where the virus has a replication rate of over 10¹¹ viral copies per day, the assisted flow rate can be selected to provide a T_{90%} in under 1 hour. In other embodiments, the assisted flow rate is selected such that the clearance rate of the assisted flow device relative to the same or substantially similar device without assisted flow is, is about, is greater than, is greater than about, 1.25, 1.50, 1.75, 2.0, 2.25, 2.50, 2.75, 3.0, 3.5, 4.0, 4.5, 5.0, 5.5, 6.0, 6.5, 7, 8, 9, 10, 11, 12, 13, 14, 15, 20, 25, 30, 35, 40, 45, or 50, or a range defined by any two of these values. In another embodiment, the assisted flow rate is selected such that the T_{90%} is reduced compared to the same or substantially similar unassisted device by, by about, by at least, by at least about, 5, 10, 15, 20, 25, 30, 35, 40, 45, 50, 55, 60, 65, 70, 75, 80, 85, 90, 95 or 99%, or a range defined by any two of these values. In other embodiments, the assisted flow device is configured such that a log plot of the percentage contaminant remaining versus time is linear, or approximately linear from 100% contaminant remaining to a value of percent remaining of, of about, of less than, of less than about, 40, 35, 30, 25, 20, 15, 14, 13, 12, 11, 10, 9, 8, 7, 6, 5, 4, 3, 2, or 1%, or a range defined by any two of these values. After the plasma component has passed through the affinity-binding medium, the treated plasma can be mixed with the cellular component and ultimately be returned to the patient, or stored separately.

Although illustrated within the context of a lectin-based binding medium for removing glycosylated viral particles, embodiments of the invention can also be used with any other contaminant-removing plasmapheresis system for which increased clearance rates and efficiency are desirable. For example, embodiments can be used with plasmapheresis systems comprising other binding materials for removing contaminants, such as activated charcoal as a binding agent for removing chemotherapeutic agents. It will be understood by those skill in the art that numerous and various modifications can be made without departing from the spirit of the present invention. Therefore, it should be clearly understood that the forms of the invention described herein are illustrative only and are not intended to limit the scope of the invention.

The particular features described in the embodiments above are not limited to the embodiments in which they are described, but can be combined with any of the embodiments of the disclosed invention. The following examples are presented to illustrate embodiments of this invention and are not intended to be restrictive.

### Example 1

Materials: GNA was obtained from Vector Labs (Burlingame, Calif.). Polysulfone hollow fiber plasma separator columns were PS20's purchased from Medica srl, (Medollo, Italy) and Minntech Wide Pore Plasma Separators (Minneapolis Minn.). Aldehyde coupling buffer containing 50 mM NaCNBH₄ came from Sigma (St Louis, Mo.). Green fluorescent latex beads, 100 nm were from Duke Scientific (Fremont Calif.); Mannan from S. cerevisiae # M7504 was from Sigma (St. Louis, Mo.). Plasma was obtained from the San Diego Blood Bank (San Diego, Calif.). All other chemicals used were reagent grade or higher.

Preparation of GNA Covalently Coupled to Diatomaceous Earth: Affinity matrices were prepared using highly purified, γ-aminopropyl-triethoxysilylated diatomaceous earth (200-300 micron) (Chromosob GAW 60-80 mesh; Celite Corp, Lompoc, Calif.). GNA (2 mg/ml) was first dialyzed against 100 volumes of cold PBS (2×4 hr; 1×16 hr) at 4° C. The GNA was then coupled to the matrix using glutaraldehyde and borohydride reduction to a stable imine. In a typical coupling, 1 gram of the γ-aminopropyl Chromosorb Celite was reacted with 5% aqueous glutaraldehyde for 1 hour in the presence of 50 mM NaCNBH₄ (Aldehyde coupling buffer; Sigma, St Louis, Mo.). After brief washing, dialyzed GNA (1 mg/ml in 1 ml PBS) was mixed with the aldehyde derivatized diatomite and reacted overnight at 4° C. in the presence of 50 mM NaCNBH₄. The GNA resin was then washed with PBS, water and ethanol and air-dried to constant weight at 40° C. in a laboratory oven. PBS washes were continued until no A280 nm was observed. Water washes were done until conductivity was reduced at least 10 fold. Ethanol was used as a single wash to remove residual water to enhance drying. The resin was dried for about 48 hours until the weight of the GNA Celite was stable. Ethanol treatment was not found to reduce the activity of the bound GNA.

Preparation of Mannan Coated Fluorescent Latex Beads: Mannan is a mannose polymer that imitates high mannose polysaccharides found on viral envelope glycoproteins. When coated onto 100 nm latex beads, the complex is very nearly the same size and shape as enveloped viruses like HIV (110 nm). The beads were prepared by adding 5.56 ml of G100 beads (10¹⁴ beads) into 5 ml of 1×PBS containing 10 mg yeast mannan. The solution was then allowed to stand overnight at room temperature. Colorimetric tests for free sugars were negative after reaction, and thus, adsorption appears to be quantitative.

Preparation and Testing of Affinity Hemodialysis cartridges: The viral HEMOPURIFIER® affinity device was made by dry filling with GNA Chromosorb into the outside compartment of a hollow-fiber dialysis column using a funnel. The external compartment of the cartridge was then sealed. GNA affinity cartridges were prepared using sterile Plasmart 20 plasma separators (200 nm pore size, 0.3 mm id polysulfone hollow-fibers) from Medica SRL, (Medollo, Italy) or with Minntech Wide Pore Plasma Separators (200 nm pore size, 0.28 mm id polysulfone hollow fibers) (Minntech, Minneapolis Minn.). The PS20 Cartridges were charged with 15 gm of affinity matrix through the dialysate ports. PS20 (neonatal) cartridges operating at 150 ml/min on 1 L of bead solution (10⁹ beads/ml in PBS containing 10% human plasma) were used. The Minntech cartridge data is included for comparison. The Minntech plasma separators were used in conjunction with a separate column containing 30 gm GNA Chromosorb affinity matrix. For the Minntech experiment the concentration of beads was 10¹⁰/ml. Clearance rates to achieve 90% clearance were compared among three different configurations: Starling flow alone (a standard HEMOPURIFIER® affinity device sealed cartridge with no external pump), an internal cartridge assisted flow configuration (a standard HEMOPURIFIER® affinity device with 25% assisted flow and an internal affinity matrix), and an external cartridge assisted flow configuration (in which the GNA affinity matrix has been removed from the plasmafilter and placed in a separate external cartridge).

Bead Transport Testing: The affinity cartridges were attached to a Cobe C3 hemodialysis machine using a standard dialysis blood tubing set. The C3 was modified to prevent dialyzate flow with the normal dialyzate circuit shunted to waste and the heater disconnected. The cartridges were washed with 1 liter of sterile PBS to remove bubbles from the lines, then slowly primed with 10% plasma solution containing 10⁹ to 10¹⁰ mannan coated beads/ml displacing the PBS wash solution to waste. At the start of the procedure, the dialysis pump was set to the appropriate rate (150 ml/min for the PS20 and 400 ml/min for the Minntech Plasma Separator). For unassisted (Starling) flow, no external pumps were used. For assisted flow, the external pump (Masterflex) was set to flow at 25% of the external flow rate. At time zero, the both the dialysis pump and the external pump (where applicable) were started and 3 ml samples were taken at intervals from the reservoir and at various points around the fluid circuit (pre and post the affinity matrix). At the end of each run, the samples were read in an Aminco Bowman spectrofluorometer (Eₓ 430 nm; Eₘ 480 nm) and the bead concentration calculated against fluorescent latex bead concentration standards.

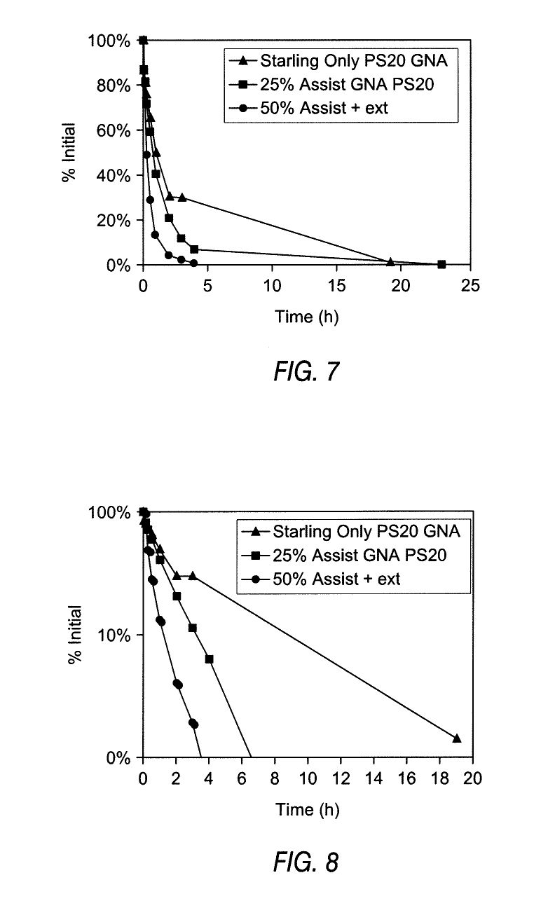

Results and Discussion: Cartridge sizes were normalized for direct comparison in three different flow configurations. It was determined that increasing flow through the shell side of a cartridge using an assist pump increases the rate of bead capture. In addition, increasing the cartridge size also increased the clearance rate. Surprisingly, assisted flow also strongly increased the time that bead capture remains linear in a log plot. FIGS. 7 and 8 shows the comparative rates of clearance, over time, of G100 mannan coated bead from 10% plasma using the PS20 cartridges in a Starling flow configuration, a 25% assisted flow in the (internal) configuration, and 50% assisted flow with the affinity matrix in an external cartridge. FIG. 7 shows a linear plot while FIG. 8 shows the log plot of the same data. As shown in FIG. 7, over 90% of the reaction is monophasic for the two assisted flow cases and is biphasic for the Starling flow. FIG. 7 also shows that increasing fluid flow through the hollow-fibers and into contact with the affinity matrix substantially increases the rate of bead clearance. (Conditions: 10⁹ mannan coated G100 beads per ml in 1 L 10% plasma. Qb=150 ml/min Qs=75 ml/min for 50% and 38 ml/min for 25% assisted flow. The 50% assist+ext refers to a system in which the GNA GAW (4-18-07) affinity matrix has been put into an external cartridge GNA vs. the Starling Flow and the 25% assist GNA PS20 where the GNA affinity matrix is packed into the extralumenal space of the PS20 cartridge. All curves are corrected for background non-binding beads averaging about 8% of total beads. These data show that pump assisted flow through the HEMOPURIFIER® affinity device cartridge enhances the removal of 100 nm particles. Virus and protein removal would thus be similarly enhanced using pump assisted flow.

TABLE I
Clearance Rates for 100 nm Mannan Coated Beads
Comparing Starling Flow with Assisted Flow

| | | | | | | Clearance | |
|---|---|---|---|---|---|---|---|
| | | Membrane Surface | Affinity | Flow Rate (ml/min) | | Time (h) | Relative |
| Sample | Cartridge | Area (m²) | Matrix | Lumenal | Shell | t_{90%} (hr) | Rate |
| Starling | PS20 | 0.15 | Internal | 150 | <8% | 8.8 | 1.0 |
| 25% Assist | PS20 | 0.15 | Internal | 150 | 38 | 3.2 | 2.8 |
| 25% Assist* | PS20 | 0.15 | External | 150 | 38 | 3.4 | 2.6 |
| 50% Assist | PS20 | 0.15 | External | 150 | 75 | 1.7 | 5.2 |
| 25% Assist | Minntech | 1 | External | 400 | 100 | 0.27 | 32.6 |
| 25% Assist* | Minntech | 1 | External | 150 | 38 | 0.72 | 12.2 |

Notes to Table I:
The volume of the reservoir was 1 liter. In all cases except Minntech, the concentration of 100 nm mannan coated fluorescent latex beads was 10⁹ beads/ml dissolved in PBS containing 10% human plasma. For the Minntech experiment the concentration of beads was 10¹⁰/ml.
*Calculated to adjust for flow rate.

As shown in Table I, the relative rates of mannan bead capture were Starling flow (1), internal matrix @ 25% assisted flow (2.8) and external matrix at 25% assisted flow (2.6). Increasing the assisted flow to 50% of total flow volume with the external matrix configuration increased the relative removal rate to 5.2 (relative to Starling flow alone). Similar values were obtained for 99% clearance as well; however, the 90% clearance rates are more biologically relevant in the absence of well defined first order kinetics.

When a larger cartridge (Minntech) was used at the same flow rates as those used with the PS20, the rate of clearance increased to 12 times faster than Starling flow alone in the PS20. This clearance rate corresponds to a 90% clearance time of 16 minutes when operated at 400 ml/min. In comparison, published data for clearance of 110 nm HIV particles using a GNA HEMOPURIFIER® affinity device cartridge typically show a 50% clearance time of 2 to 3 hours, comparable to the T_{1/2} observed here with Starling flow alone.

To put this in perspective, 50% assisted flow in Table I represents the clearance of 9×10¹² virus sized beads in 1.7 hours. In vivo, the device would be capable of clearing the average daily production of HCV (10¹¹ to 10¹² viral copies per day) in less than 1 hour. For slower growing viruses, such as HIV (10⁹ to 10¹⁰ copies/day), the device would be even more efficient. Taken together with the results of Examples 2 and 3 discussed below, these results indicate that a pump-assisted lectin affinity hemopurification device might be expected to safely reduce viral load in a patient exposed to a blood borne enveloped viral pathogen.

Conclusions: Using pump assisted flow increased the rate of virus size particle removal up to 5.2 fold over Starling flow alone, and surprisingly maintained that high removal rate up to the points of 90% and even 99% bead clearance.

### Example 2

One common limitation to changing the structure of a known hemodialysis cartridge is hemolysis. Hemolysis due to the rupture of red blood cells passing through any blood handling device (e.g. blood pumps, dialysis cartridges) is one of the most serious problems in any blood handling procedure. To determine hemolysis levels, blood is analyzed spectrophotometrically at 414 nm for the release of hemoglobin (Hb) into the plasma. Values of plasma free hemoglobin (PFH), an indicator of hemolysis, are then calculated from the formula PFH (gm/L)=(A_{414 nm}*64,500)/524,280).

One method of determining a clinically acceptable level of hemolysis in a blood treatment device is to compare the device's hemolysis rate (that is, the device's rate of Hb release) with in vivo PFH clearance rates. For an average 70 kg male with 4.6 L of blood and normal liver function, PFH is cleared in vivo at the rate of 14 gm Hb per day (that is, 333 mg/dL/day). A hemolysis rate of less than 10% of the amount that a normal, healthy human is capable of clearing in a 24 hour period is a clinically acceptable rate. Thus, for a 70 kg male, an acceptable hemolysis rate is below 33 mg/dL.

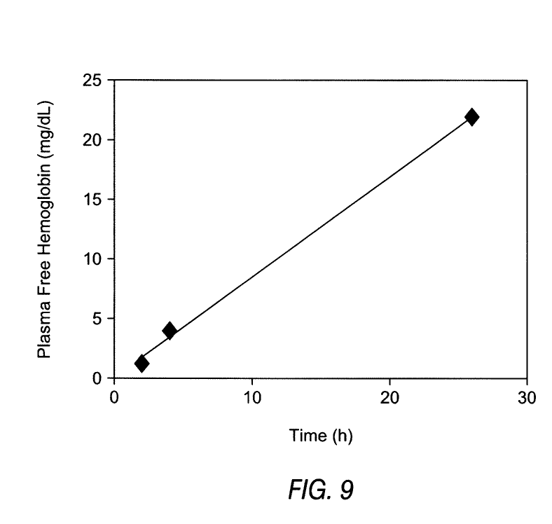

In this experiment, 400 ml of fresh human blood (San Diego Blood Bank; HCT 50%) was circulated over a HEMOPURIFIER® affinity device containing GNA for up to 26 hours using a Cobe C3 dialysis machine at room temperature. Blood samples were removed at 0, 2, 4 and 26 hours and the plasma isolated by centrifugation for 10 minutes in a clinical centrifuge. Hemoglobin (Hb) concentration was measured by absorbance at 414 nm using a molar extinction coefficient of 524,280 and a hemoglobin molecular weight of 64,500 daltons. By this measurement, Hb was released at a linear rate over the 26 h measurement period. PFH was determined to be 23 mg/dL after 26 hr of treatment with a blood flow rate of 300 ml/min (FIG. 9).

Clearance of 333 mg/dL/day is expected for a normal 70 kg man. This value is 13×larger than the approximately 25 mg PFH/dL measured for the HEMOPURIFIER® affinity device. These results indicate that, in vivo, acceptable levels of hemolysis may be expected with the HEMOPURIFIER® affinity device with Starling flow alone.

### Example 3

To investigate the potential for increased hemolysis as a result of pumping plasma out of the blood, 500 ml of human blood was circulated over the cartridge with Starling flow alone and with 25% pump assisted flow. The results are given below in Table II. Hemolysis was measured by determining free hemoglobin circulating within the plasma (PFH). No significant difference was observed under standard operating conditions; hemolysis was in fact slightly decreased over that observed with Starling flow alone.

TABLE II
Hemolysis Study on PS20 HEMOPURIFIER ® affinity device at 6 Hours

Plasma free
Hb (mg/dl)

| | Blood Flow | Observed | Predicted |
|---|---|---|---|
| Setup | (ml/min) | (PS20) | (PS60) |
| Unassisted | 120 | 24.5 | 7.8 |
| Starling Flow | | | |
| 25% Assisted Flow | 120 | 20 | 6.4 |

Conclusion: The data show that PFH went up to 24.5 mg/dL in 6 hours of recirculation using Starling flow alone. In contrast, the 25% pump assisted flow configuration yielded only 20 mg/dL PFH in the same time period. From these data it was estimated that the adult size cartridge (PS60) operating on 4.2 L blood at 400 ml/min would generate between 6.4 and 7.8 mg/dL PFH in 6 hours. This indicates that the device in either configuration would be within safe operating limits with respect to hemolysis

### Example 4

In this experiment, full sized cartridges (Medica PS60) operating on 1 liter of blood, in various configurations and at various flow rates, were used to investigate hemolysis rates.

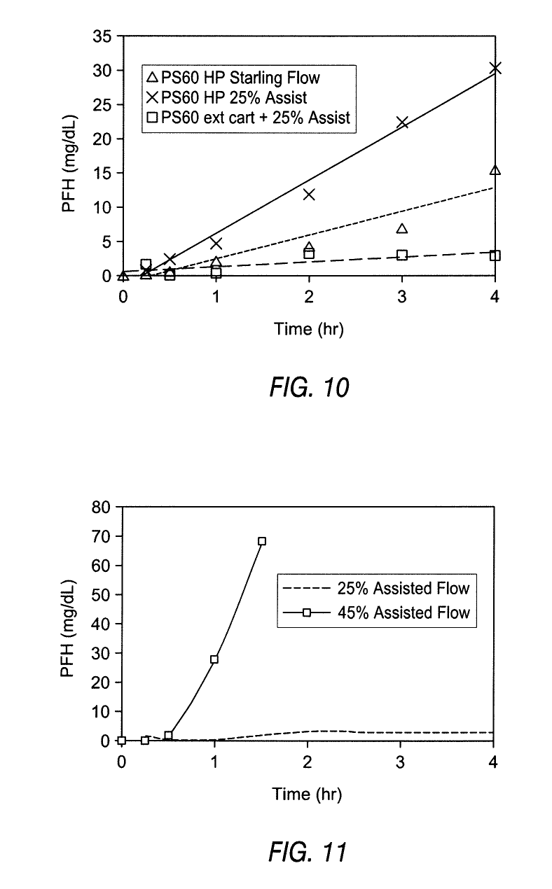

FIG. 10 compares the observed hemolysis rate for a standard PS60 GNA HEMOPURIFIER® affinity device (with Starling flow alone and with 25% assisted flow) to the hemolysis rate for an external cartridge configuration (also with 25% assisted flow). (Conditions: Qb=400 ml/min, Qs=100 ml/min or Starling Flow. Volume of reservoir=1 liter of blood (human or bovine)). PFH was calculated from A414 nm and corrected for initial (background) hemolysis divided by 4 to correct to 4 liters of blood in the reservoir. The test was run for four hours, which is a typical treatment time for dialysis patients. The results indicate that after 4 hours at a blood flow rate of 400 ml/min and a plasma flow in the shell side of the cartridge of 100 ml/min (that is, with 25% assisted flow), PFH reached a value of 30 mg/dl (assuming a blood volume of 4 L similar to that of an adult human). For the GNA HEMOPURIFIER® affinity device operated in Starling Flow mode, the 4 hour PFH value was 12 mg/dl. In comparison, the external cartridge configuration, with 25% assisted flow, resulted in a PFH of 3.0 mg/dl (a 10 fold reduction from the internal cartridge configuration).

FIG. 11 illustrates the effect on hemolysis of varying the assisted flow rate in the external cartridge configuration. (Qb=400 ml/min, Qs=100 ml/min (25%) and 180 ml/min (45%). Reservoir=1 liter of bovine blood. PFH calculated as in FIG. 10.) As shown in FIG. 11, increasing the assisted flow from 100 to 180 ml/min (25% to 45%) caused a marked increase in hemolysis. At 45% assisted flow, PFH levels reached 68 mg/dl in 90 minutes compared to ˜2 mg/dL at the 25% assisted flow rate. Thus, increasing the assisted flow rate 1.8 fold caused a 34 fold increase in hemolysis.

Results and Discussion: Hemolysis tests using full sized PS60 cartridges showed that over a 4 hour period, the Starling flow configuration and both 25% assisted flow configurations resulted in plasma free hemoglobin levels (PFH)≦30 mg/dL, well below the toxic level of 130 mg/dL. The best results were obtained using the external matrix configuration with 25% assisted flow, where PFH was 3 mg/dL after 4 hours (approximately 10 times less than for the internal affinity matrix configuration). Increasing assisted flow to 45% in the external cartridge configuration, however, caused an undesirable increase in PFH.

### Example 5

An activated charcoal plasmapheresis system includes a plasma separator cartridge having activated charcoal disposed within the plasma flow path, preferably just outside a separation membrane and within the plasma separator cartridge. An assist pump is connected in fluid communication with the plasma flow path, external to the separation cartridge. The assist pump is configured to pump plasma through the plasma flow path, out of the separation cartridge, at an assisted flow rate between 10% and 40% of the fluid flow rate of blood into the plasma separator. Downstream of the plasma separation cartridge and the assist pump, the plasma flow path preferably reconnects with the main flow path of blood flowing through the plasma separator.

In actual practice, blood from an individual having undergone a chemotherapeutic treatment is pumped into the plasma separator cartridge at up to 400 ml/min. Samples are collected prior to the entering and immediately after leaving the cartridge. The amount of chemotherapeutic agent in each collected sample can be determined by LC or GC MS.

Importantly, the chemotherapeutic agent is captured more efficiently than in a system based on Starling flow alone. Clearance rates are preferably increased significantly (e.g., as much as 2-fold) as compared to systems not having an assist pump disposed along the plasma circuit, without causing a marked increase in hemolysis rates. Accordingly, such a system can be used to advantage in vivo to clear chemotherapeutic agents from blood of a patient having undergone particularly high-dose chemotherapy, such as localized high-dose chemotherapeutic treatment of a specific organ.

1. An extracorporeal blood treatment apparatus comprising:
a separator comprising a cartridge surrounding at least one hollow fiber membrane, the hollow fiber membrane having a lumen, the cartridge and the hollow fiber membrane defining an extralumenal space there between, the separator having an inlet port and an outlet port in fluid communication with the lumen, and at least one plasma port in fluid communication with the extralumenal space,
wherein the separator is configured to allow a plasma component of blood passed through the lumen to pass through the hollow fiber membrane and into the extralumenal space while preventing a cellular portion of blood passed through the lumen to pass through the hollow fiber membrane and into the extralumenal space;
an affinity medium disposed external to the hollow fiber membrane, the affinity medium being configured to bind at least one selected contaminant; and
a plasma pump in fluid communication with the plasma port and configured to pump the plasma component at an assisted flow rate, wherein the plasma pump creates a negative pressure in the extralumenal space thereby increasing the flow rate of the plasma component across the membrane from the lumen to the extralumenal space, and wherein the assisted flow rate is selected to increase a clearance rate of the apparatus by at least two times over a clearance rate of the apparatus without the plasma pump.

2. The extracorporeal blood treatment apparatus of claim 1, wherein the assisted flow rate is between 10% and 40% of a blood flow rate into the inlet port.

3. The extracorporeal blood treatment apparatus of claim 1, wherein the affinity medium comprises lectin molecules.

4. The extracorporeal blood treatment apparatus of claim 3, wherein the lectin molecules are selected to bind to high mannose glycoproteins.

5. The extracorporeal blood treatment apparatus of claim 3, wherein the lectin molecules are immobilized within the extralumenal space.

6. The extracorporeal blood treatment apparatus of claim 3, wherein the lectin molecules are disposed in an affinity filter in fluid communication with the plasma port and plasma pump.

7. An extracorporeal blood treatment apparatus comprising:
a separator comprising a cartridge surrounding a porous separation membrane, the separation membrane configured to allow passage of a plasma component and prevent passage of a cellular component of blood passed through the separator, the separation membrane separating a main flow path of the apparatus from a plasma flow path of the apparatus;
an affinity medium disposed within the plasma flow path and configured to bind at least one selected contaminant; and
a plasma pump disposed along the plasma flow path and configured to pump the plasma component at an assisted flow rate, wherein the plasma pump creates a negative pressure in the extralumenal space thereby increasing the flow rate of the plasma component across the membrane from the lumen to the extralumenal space, and wherein the assisted flow rate is selected to reduce a T_{90%} of the apparatus by at least 50% as compared to a T_{90%} of the apparatus without the plasma pump.

8. The extracorporeal blood treatment apparatus of claim 7, wherein the assisted flow rate is between 10% and 40% of a blood flow rate into the separator.

9. The extracorporeal blood treatment apparatus of claim 7, wherein the assisted flow rate is approximately 25% of a blood flow rate into the separator.

10. The extracorporeal blood treatment apparatus of claim 7, wherein the affinity medium is disposed within the cartridge.

11. The extracorporeal blood treatment apparatus of claim 7, wherein the affinity medium is disposed in the plasma flow path external to the cartridge.

12. The extracorporeal blood treatment apparatus of claim 7, wherein the affinity medium comprises lectins selected to bind to high mannose glycoproteins.

13. An extracorporeal blood treatment apparatus comprising:
means for separating whole blood into a cellular component and a plasma component;
means for removing a selected viral pathogen from the plasma component; and
means for pumping the plasma component through the removing means at an assisted flow rate, wherein the means for pumping creates a negative pressure at the means for separating thereby increasing the flow rate of the plasma component from the means for separating, and wherein the assisted flow rate being between 10% and 40% of a fluid flow rate of whole blood flowing into the apparatus, the assisted flow rate being selected to increase a pathogen clearance rate of the apparatus by at least two times as compared to an apparatus having no such pumping means, and wherein said assisted flow rate results in hemolysis that is clinically acceptable.

14. The extracorporeal blood treatment apparatus of claim 13, wherein the removing means is disposed within the separating means.

15. The extracorporeal blood treatment apparatus of claim 13, wherein the removing means is disposed external to the separating means, in fluid communication with the pumping means in a plasma flow path.

16. The extracorporeal blood treatment apparatus of claim 13, wherein the removing means comprises lectins configured to bind to high mannose glycoproteins.

17. The extracorporeal blood treatment apparatus of claim 13, wherein the assisted flow rate is approximately 25% of the fluid flow rate of whole blood flowing into the apparatus.

18. The extracorporeal blood treatment apparatus of claim 13, wherein the pumping means is configured to pump the plasma component out of the separating means in a direction generally normal to a main flow path through the separator.

19. A method for extracorporeally treating whole blood containing a viral pathogen, the method comprising:
supplying whole blood contaminated with a viral pathogen to a separator at a whole blood flow rate so as to separate the whole blood into a cellular component and a plasma component, the separator comprising a hollow fiber membrane;
pumping the separated plasma component through an affinity medium at an assisted plasma flow rate, wherein said pumping creates a negative pressure at said separator thereby increasing the flow rate of the plasma component from the separator, and wherein the assisted flow rate being between 10% and 40% of the whole blood flow rate; and
combining the plasma component with the cellular component downstream of the affinity medium.

20. The method of claim 19, wherein the plasma component is pumped away from the separator in a direction generally normal to a main flow path through the separator.

21. The method of claim 19, wherein the viral pathogen has a viral replication rate of at least 10⁶ viral copies per day and the assisted flow rate is selected to provide a T_{90%} of not more than 2 hours.

22. In a method of reducing viral particles and lectin binding fragments thereof in the blood of an individual infected with a virus, where the method comprises the steps of obtaining blood from the individual, passing the blood through a porous hollow fiber membrane, wherein lectin molecules are immobilized within a porous exterior portion of the membrane, and wherein the lectin molecules bind to high mannose glycoproteins, collecting pass-through blood, and reinfusing the pass-through blood into the individual, the improvement comprising:
separating the blood into a plasma component traveling in a plasma flow path and a cellular component traveling in a main flow path, the hollow fiber membrane separating the main flow path from the plasma flow path;
providing a plasma pump along the plasma flow path wherein said plasma pump creates a negative pressure at the hollow fiber membrane thereby increasing the flow rate of the plasma component across the membrane from the lumen of the fiber to the porous exterior portion; and
pumping the plasma component at an assisted flow rate selected to reduce a T_{90%} of the method by at least 50% as compared to a T_{90%} of the method without the plasma pump.

23. The improvement of claim 22, wherein the assisted flow rate is between 10% and 40% of a blood flow rate into the porous hollow fiber membrane.

24. The improvement of claim 22, wherein the virus has a viral replication rate of at least 10⁶ viral copies per day and the assisted flow rate is selected to provide a T_{90%} of not more than 2 hours.
